(19) United States
(12) Patent Application Publication
(10) Pub. No.: US 2023/0265614 A1
(43) Pub. Date: Aug. 24, 2023

(54) WATER INSOLUBLE, HIGH MELTING POINT SACCHARIDE FATTY ACID ESTERS (SFAE)

(71) Applicant: GREENTECH GLOBAL PTE. LTD., Singapore (SG)

(72) Inventors: Jonathan SPENDER, Enfield, ME (US); Michael Albert Bilodeau, Clermont, FL (US); Samuel Mikail, Singapore (SG)

(73) Assignee: GREENTECH GLOBAL PTE. LTD., Singapore (SG)

(21) Appl. No.: 17/990,151

(22) Filed: Nov. 18, 2022

Related U.S. Application Data
(60) Provisional application No. 63/264,321, filed on Nov. 19, 2021.

(51) Int. Cl. D21H 21/16; D21H 19/28

(52) U.S. Cl. CPC D21H 21/16; D21H 19/28

(57) ABSTRACT

Methods of treating materials, such as cellulose-based materials, to provide barrier properties like water resistance and lipid resistance (OGR), separately or in combination, and particularly at high temperatures, by using bio-based coatings and/or compositions containing a water insoluble, high melting point saccharide fatty acid ester and products obtained by the methods.

### FIELD OF THE DISCLOSURE

The present disclosure relates generally to methods of treating materials, such as cellulose-based materials, to provide new and/or improved properties, such as improved barrier properties like water resistance and lipid resistance (OGR), separately or in combination, and particularly at high temperatures, by using bio-based coatings and/or compositions containing a water insoluble, high melting point saccharide fatty acid ester (referred to herein as a “specific SFAE”) and products obtained by the methods.

### BACKGROUND OF THE DISCLOSURE

Cellulosic materials have a wide range of applications in industry as bulking agents, absorbents, and printing components. Their employment is preferred to that of other sources of material for their high thermal stability, good oxygen barrier function, and chemical/mechanical resilience (see, e.g., Aulin et al., Cellulose (2010) 17:559-574; herein incorporated by reference in its entirety). Of great relevance is also the fact that these materials are fully biodegradable once dispersed in the environment, and that they are generally regarded as nontoxic. Cellulose and derivatives thereof are the material of choice for environmentally friendly solutions in applications such as packaging for foodstuff and disposable goods

The many advantages of cellulose are nonetheless countered by the hydrophilicity/lipophilicity of the material, which shows a high affinity for water/fats and is easily hydrated (see, e.g., Aulin et al., Langmuir (2009) 25(13):7675-7685; herein incorporated by reference in its entirety). While this is a benefit for applications such as absorbents and tissues, it becomes an issue when the safe packaging of watery/lipid containing materials (e.g., foodstuffs) is required. Long term storage of food, especially ready-made meals which contain a significant amount of water and/or fat, is made problematic in cellulose trays, for example, as they would first become soggy and then ultimately fail. Further, multiple coatings may be required to offset low efficiency of maintaining sufficient coating on the cellulosic surface due to the high relative porosity of the material, resulting in increased costs.

This problem is usually addressed in the industry by coating the cellulose fiber with some kind of hydrophobic organic material, such as fluorocarbons, waxes, and silicones, which would physically shield the underlying hydrophilic cellulose from the water/lipids in the contents, including the prevention of wicking in the fiber interstices, grease flowing into creases, or allowing the release of attached materials. For example, materials such as PVC/PEI/PE, and paraffin wax are routinely used for this purpose and are physically attached (i.e., spray coated or extruded) on the surfaces to be treated.

Industry has utilized compounds based on fluorocarbon chemistry for many years to produce articles having improved resistance to penetration by oil and grease, due to the ability of fluorocarbons to lower the surface energy of the articles. One emerging issue with the use of perfluorinated hydrocarbons is that they are remarkably persistent in the environment. The EPA and FDA have recently begun a review of the source, environmental fate, and toxicity of these compounds. A recent study reported a very high (>90%) rate of occurrence of perfluorooctane sulfonate in blood samples taken from school children. The expense and potential environmental liability of these compounds has driven manufacturers to seek alternative means of producing articles having resistance to penetration by oil and grease.

While lowering the surface energy improves the penetration resistance of the articles, lowering the surface energy also has some disadvantages. For example, a textile fabric treated with a fluorocarbon will exhibit good stain resistance; however, once soiled, the ability of cleaning compositions to penetrate and hence release the soil from the fabric may be affected, which can result in permanently soiled fabrics of reduced useful life. Another example is a greaseproof paper which is to be subsequently printed and/or coated with an adhesive. In this case the requisite grease resistance is attained by treatment with the fluorocarbon, but the low surface energy of the paper may cause problems related to printing ink or adhesive receptivity, including blocking, back trap mottle, poor adhesion, and register. If a greaseproof paper is to be used as a pressure sensitive label having an adhesive applied on one side, the low surface energy may reduce the strength of the adhesion. To improve their printability, coat-ability or adhesion, the low surface energy articles can be treated by post forming processes such as corona discharge, chemical treatment, flame treatment, or the like. However, these processes increase the cost of producing the articles and may have other disadvantages.

It would be desirable to design a “green,” biobased coating which is hydrophobic, lipophobic and compostable, including a base paper/film that would allow for keeping coatings on the surface of said paper and preventing wicking into the fiber interstices, or reducing sticking of materials to the cellulosic surface, at reduced costs, without sacrificing biodegradability and/or recyclability.

Another problem is that synthetic films, such as plastic bags, plastic wraps, plastic containers, etc., are often permeable and require one or more coating layers to achieve oil and grease resistance and/or water resistance, and/or to reduce gas permeability. Again, fluorocarbon- and/or petroleum-based coatings are typically used to provide the synthetic film with the desired barrier properties.

Another problem is that conventional coatings for imparting hydrophobic and/or lipophobic barrier properties, including the fluorocarbon and petrochemical coatings noted herein, is that they tend to perform poorly at the folds, creases, and the like of the article coated with the material. Specifically, the article typically has inferior water resistance and/or grease resistance at these locations. Such a “grease creasing effect” may be defined as the sorption of grease in a paper structure that is created by folding, pressing or crushing said paper structure. A conventional solution to the grease creasing effect is to add a latex, a butadiene, or similar resin to the coating to achieve improved coating coverage at these locations. However, with this conventional solution, the water resistance and/or oil and grease resistance of these locations may still be inferior to the flat portions of the article; this conventional solution increases cost by the addition of the resin component; and this conventional solution is not entirely biobased, since the latex and butadiene may either be synthetic and/or not easily recyclable. Accordingly, there is room for improving the barrier properties of three-dimensional objects having complex or simple shapes with folds, creases and the like.

U.S. Pat. No. 10,730,959, which issued Aug. 4, 2020 (hereinafter “the 959 patent”), which is incorporated herein by reference in its entirety, discloses tunable methods of treating a substrate, in particular cellulosic materials, with a composition that provides increased hydrophobicity and/or lipophobicity without sacrificing the biodegradability thereof. For example, the 959 patent discloses methods of binding of saccharide fatty acid esters (or “SFAE”) on cellulosic materials to provide treated materials that display higher hydrophobicity, lipophobicity, barrier function, and mechanical properties.

U.S. Pat. Application Publication No. 2021/0347999, which published Nov. 11, 2021, (hereinafter “the 999 publication”), which is incorporated herein by reference in its entirety, also discloses tunable methods of treating cellulosic materials with a composition that provides increased barrier properties, such as water resistance and/or OGR resistance, without sacrificing the biodegradability thereof. For example, the 999 publication discloses formulations containing blends of glycerides and/or fatty acid salts, optionally together with a SFAE, for imparting a cellulosic material with water and/or OGR resistance, or for providing the function of an emulsifier.

PCT/US2020/014923 (hereinafter “the ‘923 application”), which is incorporated herein by reference in its entirety, discloses methods of treating fibrous cellulosic materials with sucrose fatty acid ester containing particles (carrier systems) that allow for modifications of surfaces, including making such surfaces water resistance and/or oil/grease resistance. The methods as disclosed provide combining at least one SFAE with a polymer (e.g., latexes) to form micellular particles and applying such particles to substrates including fibrous cellulose-based materials (e.g., pulp) to form, inter alia, molded products. Compositions comprising combinations of SFAE, a latex and optionally a mineral or other additives are also disclosed.

US 16/568,953 (hereinafter “the ‘953 application”), which is incorporated herein by reference in its entirety, discloses tunable methods of treating cellulosic materials with a barrier coating comprising a prolamin and at least one polyol fatty acid ester that provides increased oil and/or grease resistance to such materials without sacrificing the biodegradability thereof. The methods as disclosed provide for adhering of the barrier coating on articles including articles comprising cellulosic materials and articles made by such methods. The materials thus treated display higher lipophobicity and may be used in any application where such features are desired.

US 16/456,499 (hereinafter “the ‘499 application”), which is incorporated herein by reference in its entirety, discloses tunable methods of treating cellulosic materials with a barrier coating comprising at least two polyol and/or saccharide fatty acid ester that provides increased water, oil and grease resistance to such materials without sacrificing the biodegradability thereof. The methods as disclosed provide for adhering of the barrier coating on articles including articles comprising cellulosic materials and articles made by such methods. The materials thus treated may display higher hydrophobicity and lipophobicity and may be used in any application where such features are desired.

US 16/456,433 (hereinafter “the ‘433 application”), which is incorporated herein by reference in its entirety, discloses methods of treating cellulosic materials with compositions that allow greater retention of inorganic particles on cellulosic substrates. The methods as disclosed provide combining SFAE with such inorganic particles and applying such combinations on cellulosic materials to eliminate or reduce the use of retention aids or binders for filler in the paper making process. Compositions comprising such combinations of SFAE and inorganic particles are also disclosed.

On the other hand, the inventors determined that migration may occur when a cellulosic material, which has been derivatized for improved barrier properties with a barrier formulation based on conventional SFAEs, is contacted by a relatively hot material. For example, when the treated cellulosic material is a food packaging, and the packaging comes in contact with foodstuff having a relative high temperature, the SFAE may melt and migrate into the foodstuff or otherwise relocate, resulting in decreased barrier properties and possible leakage.

Accordingly, there is still a need for “green,” bio-based formulations that provide improved barrier properties for cellulose-based materials, particularly at high temperature.

In addition, conventional SFAEs are also highly anionic in aqueous media and are repelled from the surfaces of cellulose fiber in aqueous slurries, which are also anionically charged. This may present challenges to retain the SFAE in the fibrous web or article formed from the draining of the fibers and SFAE from a slurry. In this regard, a conventional solution may be to use a retention aid, such as a charged polymer. However, this may have drawbacks, such as the desire for a “green” process, and may complicate the process by requiring an additional step or material.

### SUMMARY OF THE DISCLOSURE

Based on diligent effort by the inventors, the present disclosure provides barrier formulations and methods using a water insoluble and high melting point saccharide fatty acid ester (hereinafter “the specific SFAE”), which can impart improved water resistance and/or improved oil and grease resistance (OGR) to cellulosic materials, and maintain this improved barrier property at relatively high temperatures. The present disclosure also provides articles and products treated or made with the barrier formulations, the articles having improved properties, including but not limited to water resistance and/or OGR.

In some embodiments, the specific SFAE does not dissolve in water at 25° C.

In some embodiments, the specific SFAE has a melting point higher than 100° C. In some aspects the melting point of the specific SFAE may be 110° C. or higher, 120° C. or higher, 130° C. or higher, 140° C. or higher, or 150° C. or higher. In some embodiments, the SFAE has a melting point lower than 250° C., lower than 225° C., lower than 200° C., lower than 175° C., or lower than 150° C.

It was considered surprising, for example, given its solubility properties (that is, the specific SFAE is substantially non-polar and hydrophobic and thus insoluble in water), that the specific SFAE can provide OGR. That is, one would not expect a material having such properties to repel lipids.

In addition, given its relatively high melting point, the specific SFAE may also address the problem of migration in foodstuff packaging. That is, at the relatively high temperatures of certain foodstuffs, the specific SFAE is not likely to melt. Stated differently, the specific SFAE increases resistance to hot oil penetration, which is highly desirable in hot food packaging and reheating applications.

It was also considered surprising that a non-film-forming material, such as the specific SFAE, could be utilized to provide water resistance and OGR. Both properties, of course, are beneficial for a barrier formulation for a cellulose-based material generally, and for an article intended to contact foodstuff specifically.

In some embodiments, the present disclosure provides a method of imparting hydrophobic and/or lipophobic barrier properties to a substrate, the method including: preparing a formulation for imparting the hydrophobic and/or lipophobic barrier properties to the substrate, the formulation including an effective amount of the specific SFAE to impart the barrier property; and contacting a surface of the substrate with the formulation to impart the hydrophobic and/or lipophobic barrier properties to the substrate.

In some embodiments the method includes a step of predetermining an amount of the specific SFAE to be included in the formulation. In some aspects, the step of predetermining can be performed prior to the preparing the formulation, or can be performed prior to the contacting the surface of the substrate with the formulation. In some aspects, the step of predetermining is performed to achieve the desired effects. In some aspects, the step of predetermining is performed to achieve a desired level of water resistance and/or a desired level of OGR.

In some embodiments, the substrate contacted with the formulation is a cellulosic material, a synthetic polymeric material, or a natural or synthetic woven material. In some embodiments, the cellulosic material can be cellulose fibers, microfibrillated cellulose (MFC), nanofibrillated cellulose, cellulose nanofibers (CNF), or cellulose nanocrystals.

In some embodiments, the step of contacting the substrate with the formulation includes forming a slurry of the formulation and cellulose fiber. This can be referred to as a wet end process (i.e., prior to draining the fibers from the slurry; as opposed to coating the surface of an already formed article).

In some embodiments, the formulation can be in the form of an emulsion.

In some embodiments, the specific SFAE is present in the slurry at a concentration of at least 0.025% (wt/wt) of the total cellulose fiber present. In related aspects, the specific SFAE may be present at about 0.05% (wt/wt) to about 0.1 % (wt/wt), about 0.1% (wt/wt) to about 0.5% (wt/wt), about 0.5% (wt/wt) to about 1.0% (wt/wt), about 1.0% (wt/wt) to about 2.0% (wt/wt), about 2.0% (wt/wt) to about 3.0% (wt/wt), about 3.0% (wt/wt) to about 4.0% (wt/wt), about 4.0% (wt/wt) to about 5.0% (wt/wt), about 5.0%(wt_{/}wt) to about 10% (wt/wt), or about 10% (wt/wt) to about 50% (wt/wt) of the total fiber present.

In some embodiments, a water-soluble saccharide fatty acid ester (hereinafter “soluble SFAE”) can be added to the slurry as an emulsifier or emulsifying agent, such as to facilitate solubilizing or otherwise dispersing the water-insoluble specific SFAE in water. Thus, the soluble SFAE dissolves in water at 25° C. The soluble SFAE may be a SFAE made according to the 959 patent. The soluble SFAE may be liquid at room temperature.

In some aspects, the soluble SFAE is a sucrose ester.

In some aspects, the soluble SFAE may consist essentially of, or consist of, unsaturated fatty acid groups.

In some aspects, the soluble SFAE may be a monoester. In some aspects, the soluble SFAE may contain greater than 50% by weight, greater than 60 wt%, greater than 70 wt%, greater than 80 wt%, or greater than 90 wt% monoesters.

In some embodiments, the formulation may be, for example, a liquid system consisting essentially of, or consisting of, the specific SFAE and the soluble SFAE. That is, in some aspects, the formulation may be a liquid containing only (or materially only) the specific SFAE and the soluble SFAE.

In some embodiments, the formulation may contain a water emulsion of the specific SFAE and the soluble SFAE.

In some aspects, when the soluble SFAE acts as a solvent for the specific SFAE, the distribution of the specific SFAE throughout a fibrous web or substrate may be improved, thereby improving the efficiency of generating barrier properties.

In some embodiments, the formulation may contain the specific SFAE and an organic solvent.

In some embodiments, an article formed using the slurry possesses hydrophobic and/or lipophobic barrier properties. The article formed from the solution may include paper, paperboard, bacon board, insulating material, a carton for food storage, a compost bag, a bag for food storage, release paper such as for an adhesive such as a pressure sensitive adhesive, a shipping bag, weed-block/barrier fabric or film, mulching film, plant pots, packing beads, bubble wrap, laminates, envelops, gift cards, credit cards, gloves, raincoats, OGR paper, a shopping bag, diapers, membranes, eating utensil, a tea bag, a container for coffee or tea, a container for holding hot or cold beverages, a cup, a plate, a bottle for carbonated liquid storage, a bottle for non-carbonated liquid storage, a lid, film for wrapping food, a garbage disposal container, a food handling implement, a fabric fibre, a water storage and conveying implement, a storage and conveying implement for alcoholic or non-alcoholic beverages, an outer casing or screen for electronic goods, an internal or external piece of furniture, a curtain, upholstery, fabric, film, a box, a sheet a tray, a pipe, a tube, a water conduit, clothing, a medical device, pharmaceutical packaging, a contraceptive, camping equipment, cellulosic material that is molded, and combinations thereof.

In some embodiments, the step of contacting the substrate with the formulation includes coating the surface of the cellulose-based substrate with the formulation. In some embodiments, the specific SFAE is present on the surface of the substrate at a coating weight of at least about 0.05 g/m² on the surface of the substrate. In related aspects, the specific SFAE can be present at a coating weight of about 0.05 g/m² to about 1.0 g/m², about 1.0 g/m² to about 2.0 g/m², about 2 g/m² to about 3 g/m² on a surface of the cellulose-based material. In a related aspect, the specific SFAE may be present from about 3 g/m² to about 4 g/m², about 4 g/m² to about 5 g/m² about 5 g/m² to about 10 g/m², or about 10 g/m² to about 20 g/m².

In some embodiments, the formulation used for coating the substrate may include the soluble SFAE, or the formulation may be the liquid system which consists essentially of or consists of, the specific SFAE and the soluble SFAE.

In some embodiments, the substrate contacted with the formulation is the surface of an article selected from the group consisting of paper, paperboard, bacon board, insulating material, paper pulp, a carton for food storage, a compost bag, a bag for food storage, release paper such as for a pressure sensitive adhesive, a shipping bag, weed-block/barrier fabric or film, mulching film, plant pots, packing beads, bubble wrap, oil absorbent material, laminates, envelops, gift cards, credit cards, gloves, raincoats, OGR paper, a shopping bag, diapers, membranes, eating utensil, a tea bag, a container for coffee or tea, a container for holding hot or cold beverages, a cup, a plate, a bottle for carbonated liquid storage, a bottle for non-carbonated liquid storage, a lid, film for wrapping food, a garbage disposal container, a food handling implement, a fabric fibre, a water storage and conveying implement, a storage and conveying implement for alcoholic or non-alcoholic beverages, an outer casing or screen for electronic goods, an internal or external piece of furniture, a curtain, upholstery, fabric, film, a box, a sheet, a tray, a pipe, a tube, a water conduit, clothing, a medical device, pharmaceutical packaging, a contraceptive, camping equipment, cellulosic material that is molded, and combinations thereof.

In some embodiments, the specific SFAE may be used as a pigment (where the term pigment has its general meaning the art of papermaking). In this embodiment, a fine particle of undissolved specific SFAE may be incorporated into the fibrous web; and then the drying/forming heat and pressure during manufacturing of the cellulose-based article will melt the specific SFAE and cause it to flow and fill pores.

In some embodiments, the fatty acid chain(s) of the specific SFAE (or the soluble SFAE) is obtained from oilseeds. In other embodiments, the fatty acid chain(s) are obtained from other sources of naturally occurring, edible fats and oil.

In some embodiments, the specific SFAE has one or esterified hydroxyl groups of the saccharide moiety. In some aspects, the specific SFAE may be, for example, a monoester, diester, or triester. In some aspects, the specific SFAE may contain greater than 50% by weight, greater than 60 wt%, greater than 70 wt%, greater than 80 wt%, or greater than 90 wt% monoesters and diesters. In some aspects, the specific SFAE may contain monoesters as a major component (as used herein, major component means greater than 50 wt% in the context used). In some aspects, the specific SFAE may contain diesters as a major component.

In some embodiments, the fatty acid groups of the specific SFAE may have different carbon numbers, different degrees of unsaturation, and/or different configurations and positions of olefins. That is, when the degree of substitution is higher than one (e.g., a saccharide diester), each fatty acid group may be the same or different in one or more of these characteristics.

In some embodiments, the fatty acid group may be selected from, for example, stearate, laurate, myristate, and palmitate, but the fatty acid group is not so limited.

In some embodiments, the saccharide moiety may be, for example, one or more selected from lactose, maltose, raffinose, or trehalose, but the saccharide moiety is not so limited.

In some embodiments, the saccharide moiety may be chitosan, which is a linear polysaccharide having a cationic charge. Chitosan can be isolated from shellfish skeletons and is positively charged. Chitosan is a high molecular weight saccharide moiety, as compared to a simple disaccharide, and commercially produced chitosan may have a molecular weight of about 3,800 to about 20,000 daltons.

By utilizing cationic chitosan as the saccharide moiety of the specific SFAE, the electrostatic attraction between the thus cationic SFAE and the anionic cellulose surfaces may be increased, resulting in increased absorption and retention of the SFAE when forming paper and molded pulp products.

In some embodiments, the formulation includes two or more specific SFAE, wherein one of the specific SFAE has a disaccharide moiety, and one of the specific SFAE has a chitosan moiety. In some embodiments, this formulation may further include the soluble SFAE.

In some embodiments, the hydrophobic barrier property is imparted to the substrate by the formulation in the absence of any secondary hydrophobes.

In some embodiments, the formulation may include one or more emulsifiers or emulsifying agents. When used, a weight ratio of the specific SFAE to the one or more emulsifying agents is from about 0. 1:99.9 to about 99.0:0.1, from about 10:90 to about 90:10, from about 20:80 to 80:20, from about 35:65 to 65:35, from about 40:60 to about 60:40, or about 50:50. In some embodiments, the emulsifying agent can be selected from water, the soluble SFAE, buffers, saccharide fatty acid esters, polyvinyl alcohol (PvOH), carboxymethyl cellulose (CMC), milk proteins, wheat glutens, gelatins, prolamines, soy protein isolates, starches, acetylated polysaccharides, alginates, carrageenans, chitosans, inulins, long chain fatty acids, waxes, agar, alginates, glycerol, gums, lecithins, poloxamers, mono-, di-glycerols, monosodium phosphates, monostearate, propylene glycols, detergents, cetyl alcohol, glycerol esters, (saturated) ((poly)unsaturated) fatty acid methyl esters, and combinations thereof.

As noted above, in some embodiments, the formulation used in the methods also includes one or more of the soluble SFAE. When used, a weight ratio of the specific SFAE to the soluble SFAE is from about 0.1:99.9 to about 99.0:0.1, from about 10:90 to about 90:10, from about 20:80 to 80:20, from about 35:65 to 65:35, from about 40:60 to about 60:40, or about 50:50.

In some embodiments the method includes a step of predetermining a content of the specific SFAE to be included in the formulation. In some aspects, this step of predetermining can be performed prior to the preparing the formulation, or can be performed prior to the contacting the surface of the substrate with the formulation. In some aspects, this step of predetermining is performed to achieve the desired effects. In some aspects, the step of predetermining is performed to achieve a desired level of water resistance and/or a desired level of oil and grease resistance.

In some embodiments, the formulation used may include one or more pigments commonly used in the paper industry. The one or more pigments can be present in the formulation in a concentration of about 0. 1% to about 90% by weight based on a total weight of the formulation. In other aspects, the concentration of the pigment can be from about 1% to 10% by weight, from about 11% to 20% by weight, from about 21% to 30% by weight, from about 31 % to 40% by weight, from about 41 % to 50% by weight, 51 % to 60% by weight, 61 % to 70% by weight, 71 to 80% by weight, 81% to 90% by weight, or any other range between 0.1% to 90% by weight. The use of pigments is well known in the paper industry, and the pigment concentration can be chosen to vary the properties of the final product. In some embodiments, the one or more pigments are selected from clay, calcium carbonate, titanium dioxide, kaolin, talc, or plastic pigment.

In some embodiments, the one or more pigments are pre-treated prior to being included in the formulation. A pre-treatment can include contacting the pigment with a saccharide fatty acid ester for a sufficient length of time and temperature sufficient to bind the saccharide fatty acid ester to the pigment. For example, in one aspect, the specific SFAE may be heated and melted onto the surface of an inorganic particle / pigment. The pre-treated pigment can be included in the wet-end (e.g., added directly to the paper-making furnish) or can added to the formulations of the present disclosure.

In some embodiments, the formulation is entirely biobased. In some embodiments, the formulation does not include fluorocarbons. In some embodiments, the formulation does not include a compound obtained from petroleum. In some embodiments, the articles made using the formulation are entirely biobased.

In some embodiments, the formulation includes one or more charged polymers to aid in the retention of the specific SFAE on the substrate. The one or more charged polymers may include one or more cationic polymers, anionic polymers, nonionic polymers, and/or zwitterionic polymers. In some embodiments, the charged polymer may include a combination of a relatively low molecular weight cationic polymer and a relative high molecular weight anionic polymer. In some embodiments, the formulation does not include a charged polymer.

In some embodiments, the charged polymer consists of one or more cationic polymer. The one or more cationic polymer may include a polyacrylamide. The polyacrylamide may include polyDADMAC (poly diallyldimethylammonium chloride).

In some embodiments, the cationic polymer has a weight average molecular weight of 500,000 to 10,000,000. In some aspects, the weight average MW is 500,000 to 1,000,000, 1,000,001 to 2,000,000, 2,000,001 to 3,000,000, 3,000,001 to 4,000,000, 4,000,001 to 5,000,000, 5,000,001 to 6,000,000, 6,000,001 to 7,000,000, 7,000,001 to 8,000,000, 8,000,001 to 9,000,000, or 9,000,001 to 10,0000. In some aspects, a blend of charged polymers are used to achieve a “bimodal”-type weight average MW using a combination of charged polymers having any MW in the ranges above (e.g., a first charged polymer having a weight average MW of less than 1,000,000 used in combination with a second charged polymer having a weight average MW greater than 2,000,000; wherein the weight ratio of the first charged polymer to the second charged polymer is 10:90 to 90:10). In some embodiments, a concentration of the cationic polymer in the formulation is from about 0.01% to about 5% by weight, from about 0.01% to about 3% by weight, 0.05% to about 0.1% by weight, or from about 0.1% to about 1% by weight, or from about 1% to about 3% by weight when a total weight of the formulation is considered 100%. In some aspects, a weight ratio in the formulation of the cationic polymer to the specific SFAE is from about 0.1:99.9 to about 20:80, from 0.5:99.5 to about 15:85, from about 1:99 to about 10:90, or from about 2.5:97.5 to about 7.5:92.5.

In some embodiments, the formulation may include one or more binders selected from, for example, starch, protein, prolamine, polymers, polymer emulsions, PvOH, or combinations thereof. In some embodiments, the formulation does not contain a binder.

In some embodiments, the substrate imparted with the hydrophobic and/or lipophobic barrier properties exhibits a 3 M grease KIT test value of between about 3 and about 12. In some embodiments, the surface of the substrate imparted with the hydrophobic and/or lipophobic barrier properties exhibits a water contact angle greater 90°, greater than 100°, greater than 110°, or greater than 120°. In some embodiments, the surface of the substrate imparted with the hydrophobic and/or lipophobic barrier properties exhibits an HST value of at least 65 secs.

In some embodiments, an article is provided that is obtained by the disclosed methods and/or otherwise treated with the formulation.

Additional features and advantages of the present disclosure are described further below. This summary section is meant merely to illustrate certain features of the disclosure, and is not meant to limit the scope of the disclosure in any way. The failure to discuss a specific feature or embodiment of the disclosure, or the inclusion of one or more features in this summary section, should not be construed to limit the claims.

### DETAILED DESCRIPTION OF THE DISCLOSURE

Before the present compositions, methods, and methodologies are described in more detail, it is to be understood that the disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a specific SFAE” includes one or more SFAE, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this disclosure belongs. Any methods and materials similar or equivalent to those described herein may be used in the practice or testing of the disclosure, as it will be understood that modifications and variations are encompassed within the spirit and scope of the instant disclosure.

Unless otherwise stated, each range disclosed herein will be understood to encompass and be a disclosure of each discrete point and all possible subranges within the range.

As used herein, “about,” “approximately,” “substantially,” and “significantly” will be understood by a person of ordinary skill in the art and will vary in some extent depending on the context in which they are used. If there are uses of the term which are not clear to persons of ordinary skill in the art given the context in which it is used, “about” and “approximately” will mean plus or minus <10% of particular term, and “substantially” and “significantly” will mean plus or minus > 10% of the particular term. “Comprising” and “consisting essentially of” have their customary meaning in the art.

In embodiments, the present disclosure shows that by treating the surface of substrate, such as, for example, cellulose fibers, with a specific SFAE, the resulting surface is, inter alia, made strongly hydrophobic. For example, in the case of cellulose fiber, the cellulosic hydroxyl groups can be masked by bulky organic chains. Further, the specific SFAE, for example, once removed by bacterial enzymes, are easily digested as such. The derivatized surface of the substrate has been shown to display a great deal of heat resistance, being able to withstand temperatures as high as 250° C. and may be more impermeant to gases than the base substrate underneath. The material is therefore an ideal solution to the problem, for example, of derivatizing the hydrophilic surface of cellulose, in any embodiment in which cellulose materials may be employed, including, for example, packaging for foodstuff.

Advantages of the products and methods as disclosed herein include that the coating composition is made from renewable agricultural resources-e.g., vegetable oils; is biodegradable; has a low toxicity profile and suitable for food contact; can be tuned to reduce the coefficient of friction of the substrate (e.g., with regard to a paper/paperboard surface the treatments do not make the paper too slippery for downstream processing or end use), even at high levels of water resistance; may or may not be used with special emulsification equipment or emulsification agents; and is compatible with traditional paper recycling programs: i.e., poses no adverse impact on recycling operations, like polyethylene, polylactic acid, or wax coated papers do.

As used herein, “biobased” means a material intentionally made from substances derived from living (or once-living) organisms. In a related aspect, material containing at least about 50% of such substances is considered biobased. However, as noted above, in some embodiments the articles disclosed herein may contain up to 100% of such substances.

As used herein, “bind”, including grammatical variations thereof, means to cohere or cause to cohere essentially as a single mass, and may refer to ionic, hydrophobic, van der Waals interaction, or covalent bonding, or a combination thereof.

As used herein, “cellulosic” means natural, synthetic or semisynthetic materials that can be molded or extruded into objects (e.g., bags, sheets) or films or filaments, which may be used for making such objects or films or filaments, that is structurally and functionally similar to cellulose, e.g., coatings and adhesives (e.g., carboxymethylcellulose). In another example, cellulose, a complex carbohydrate (C₆H₁₀O₅)n that is composed of glucose units, which forms the main constituent of the cell wall in most plants, is cellulosic.

As used herein, “coating weight” is the weight of a material (wet or dry) applied to a substrate. It is expressed in pounds per specified ream or grams per square meter

As used herein, “compostable” means these solid products are biodegradable into the soil.

As used herein, “edge wicking” means the sorption of water in a paper structure at the outside limit of said structure by one or more mechanisms including, but not limited to, capillary penetration in the pores between fibers, diffusion through fibers and bonds, and surface diffusion on the fibers. In a related aspect, the glyceride and/or fatty acid salt containing formulation as described herein prevents edge wicking in treated products. In one aspect, a similar problem exists with grease/oil entering creases that may be present in paper or paper products. Such a “grease creasing effect” may be defined as the sorption of grease in a paper structure that is created by folding, pressing or crushing said paper structure.

As used herein, “effect”, including grammatical variations thereof, means to impart a particular property to a specific material.

As used herein, “hydrophobe” means a substance that does not attract water. For example, waxes, rosins, resins, saccharide fatty acid esters, fatty acid salts, glycerides having long fatty acid chains; di- and triglycerides, diketenes, shellacs, vinyl acetates, PLA, PEI, oils, fats, lipids, other water repellant chemicals or combinations thereof are hydrophobes.

As used herein, “hydrophobicity” means the property of being water-repellent, tending to repel and not absorb water.

As used herein, “lipid resistance” or “lipophobicity” means the property of being lipid-repellent, tending to repel and not absorb lipids, grease, fats and the like. In a related aspect, the grease resistance may be measured by a “3M KIT” test, a TAPPI T559 Kit test, or a Cobb oil test.

As used herein, “cellulose-containing material” or “cellulose-based material” means a composition which consists essentially of cellulose. For example, such material may include, but is not limited to, paper, paper sheets, paperboard, paper pulp, a carton for food storage, parchment paper, cake board, butcher paper, release paper/liner for a pressure sensitive adhesive, a bag for food storage, a shopping bag, a shipping bag, bacon board, insulating material, tea bags, containers for coffee or tea, a compost bag, eating utensil, container for holding hot or cold beverages, cup, a lid, plate, a bottle for carbonated liquid storage, gift cards, a bottle for non-carbonated liquid storage, film for wrapping food, a garbage disposal container, a food handling implement, a fabric fibre (e.g., cotton or cotton blends), a water storage and conveying implement, alcoholic or non-alcoholic drinks, an outer casing or screen for electronic goods, an internal or external piece of furniture, a curtain and upholstery.

As used herein, “release paper” means a paper sheet used to prevent a sticky surface from prematurely adhering to an adhesive or a mastic, such as, for example, for a pressure sensitive adhesive. In one aspect, the coatings as disclosed herein can be used to replace or reduce the use of silicon or other coatings to produce a material having a low surface energy. Determining the surface energy may be readily achieved by measuring contact angle (e.g., Optical Tensiometer and/or High Pressure Chamber; Dyne Testing, Staffordshire, United Kingdom) or by use of Surface Energy Test Pens or Inks (see, e.g., Dyne Testing, Staffordshire, United Kingdom).

As used herein “releasable” with reference to the specific SFAE means that the material, once applied, may be removed from the substrate (e.g., a cellulose-based material), such as by manipulating physical properties). As used herein “non-releasable” with reference to the specific SFAE means that the material, once applied, is substantially irreversibly bound to the substrate (e.g., cellulose-based material), such as by chemical means.

As used herein, “fibers in solution” or “pulp” means a lignocellulosic fibrous material prepared by chemically or mechanically separating cellulose fibers from wood, fiber crops or waste paper. In a related aspect, where cellulose fibers are treated by the methods as disclosed herein, the cellulose fibers themselves contain bound specific SFAE as isolated entities, and where the bound cellulose fibers have separate and distinct properties from free fibers (e.g., pulp- or cellulose fiber- or nanocellulose or microfibrillated cellulose-glyceride/fatty acid salt bound material would not form hydrogen bonds between fibers as readily as unbound fibers).

As used herein, “repulpable” means to make a paper or paperboard product suitable for crushing into a soft, shapeless mass for reuse in the production of paper or paperboard.

As used herein, “tunable”, including grammatical variations thereof, means to adjust or adapt a process to achieve a particular result.

As used herein, “water contact angle” means the angle measured through a liquid, where a liquid/vapor interface meets a solid surface. It quantifies the wettability of the solid surface by the liquid. The contact angle is a reflection of how strongly the liquid and solid molecules interact with each other, relative to how strongly each interacts with its own kind. On many highly hydrophilic surfaces, water droplets will exhibit contact angles of 0° to 30°. Generally, if the water contact angle is larger than 90°, the solid surface is considered hydrophobic. Water contact angle may be readily obtained using an Optical Tensiometer (see, e.g., Dyne Testing, Staffordshire, United Kingdom).

As used herein, “water vapour permeability” means breathability or a textile’s ability to transfer moisture. There are at least two different measurement methods. One, the MVTR Test (Moisture Vapour Transmission Rate) in accordance with ISO 15496, describes the water vapor permeability (WVP) of a fabric and therefore the degree of perspiration transport to the outside air. The measurements determine how many grams of moisture (water vapor) pass through a square meter of fabric in 24 hours (the higher the level, the higher the breathability).

In one aspect, TAPPI T 530 Hercules size test (i.e., size test for paper by ink resistance) may be used to determine water resistance. Ink resistance by the Hercules method is best classified as a direct measurement test for the degree of penetration. Others classify it as a rate of penetration test. There is no one best test for “measuring sizing.” Test selection depends on end use and mill control needs. This method is especially suitable for use as a mill control sizing test to accurately detect changes in sizing level. It offers the sensitivity of the ink float test while providing reproducible results, shorter test times, and automatic end point determination.

Sizing, as measured by resistance to permeation through or absorption into paper of aqueous liquids, is an important characteristic of many papers. Typical of these are bag, containerboard, butcher’s wrap, writing, and some printing grades.

This method may be used to monitor paper or board production for specific end uses provided acceptable correlation has been established between test values and the paper’s end use performance. Due to the nature of the test and the penetrant, it will not necessarily correlate sufficiently to be applicable to all end use requirements. This method measures sizing by rate of penetration. Other methods measure sizing by surface contact, surface penetration, or absorption. Size tests are selected based on the ability to simulate the means of water contact or absorption in end use. This method can also be used to optimize size chemical usage costs.

As used herein, “oxygen permeability” means the degree to which a polymer allows the passage of a gas or fluid. Oxygen permeability (Dk) of a material is a function of the diffusivity (D) (i.e., the speed at which oxygen molecules traverse the material) and the solubility (k) (or the amount of oxygen molecules absorbed, per volume, in the material). Values of oxygen permeability (Dk) typically fall within the range 10-150 × 10¹¹ (cm² ml O₂)/(s ml mmHg). A semi-logarithmic relationship has been demonstrated between hydrogel water content and oxygen permeability (Unit: Barrer unit). The International Organization for Standardization (ISO) has specified permeability using the S1 unit hectopascal (hPa) for pressure. Hence Dk = 10^{...11} (cm² ml O₂) /(s ml hPa). The Barrer unit can be converted to hPa unit by multiplying it by the constant 0.75.

As used herein “biodegradable”, including grammatical variations thereof, means capable of being broken down especially into innocuous products by the action of living things (e.g., by microorganisms).

As used herein, “recyclable”, including grammatical variations thereof, means a material that is treatable or that can be processed (with used and/or waste items) so as to make said material suitable for reuse.

As used herein, “Gurley second” or “Gurley number” is a unit describing the number of seconds required for 100 cubic centimeters (deciliter) of air to pass through 1.0 square inch of a given material at a pressure differential of 4.88 inches of water (0.176 psi) (ISO 5636-5:2003)(Porosity). In addition, for stiffness, “Gurley number” is a unit for a piece of vertically held material measuring the force required to deflect said material a given amount (1 milligram of force). Such values may be measured on a Gurley Precision Instruments’ device (Troy, New York).

HLB-The hydrophilic-lipophilic balance of a surfactant is a measure of the degree to which it is hydrophilic or lipophilic, determined by calculating values for the different regions of the molecule.

Griffin’s method for non-ionic surfactants as described in 1954 works as follows:

$$H L B = 20 ∗ {M}_{h} / M$$

where Mₕ is the molecular mass of the hydrophilic portion of the molecule, and M is the molecular mass of the whole molecule, giving a result on a scale of 0 to 20. An HLB value of 0 corresponds to a completely lipophilic/hydrophobic molecule, and a value of 20 corresponds to a completely hydrophilic/lipophobic molecule.

The HLB value can be used to predict the surfactant properties of a molecule:
< 10 : Lipid-soluble (water-insoluble)
> 10 : Water-soluble (lipid-insoluble)
1.5 to 3 : anti-foaming agent
3 to 6 : W/O (water in oil) emulsifier
7 to 9 : wetting and spreading agent
13 to 15 : detergent
12 to 16 : O/W (oil in water) emulsifier
15 to 18 : solubiliser or hydrotrope.

In some embodiments, the HLB values for the specific SFAE (or formulation comprising said specific SFAE) as disclosed herein may be in the lower range. In some embodiments, the HLB values for the specific SFAE (or formulation comprising said specific SFAE) as disclosed herein may be in the middle to higher ranges. In some embodiments, the HLB values for the soluble SFAE as disclosed herein may be in the lower range. In other embodiments, the HLB values for the soluble SFAE as disclosed herein may be in the middle to higher ranges.

As used herein, “SEFOSEⓇ” denotes a sucrose fatty acid ester made from soybean oil (soyate) which is commercially available from Procter & Gamble Chemicals (Cincinnati, OH) under the trade name SEFOSE 1618U (see sucrose polysoyate below), which contains one or more fatty acids that are unsaturated. As used herein, “OLEAN®” denotes a sucrose fatty acid ester which is available from Procter & Gamble Chemicals having the formula Cₙ+₁₂H₂ₙ₊₂₂O₁₃, where all fatty acids are saturated. The Examples of the 959 patent, mentioned above and incorporated herein by reference, employed SEFOSE as an SFAE for imparting barrier properties to substrates, including cellulosic materials.

As used herein, “soyate” means a mixture of salts of fatty acids from soybean oil.

As used herein, “oilseed fatty acids” means fatty acids from plants, including but not limited to soybeans, peanuts, rapeseeds, barley, canola, sesame seeds, cottonseeds, palm kernels, grape seeds, olives, safflowers, sunflowers, copra, corn, coconuts, linseed, hazelnuts, wheat, rice, potatoes, cassavas, legumes, camelina seeds, mustard seeds, and combinations thereof. The fatty acid chains of the SFAEs of the present disclosure can be oilseed fatty acids.

As used herein “wet strength” means the measure of how well a web of fibers holding paper together (or other three-dimensional, solid, cellulose-based product) can resist a force of rupture when the paper is wet. The wet strength may be measured using a Finch Wet Strength Device from Thwing-Albert Instrument Company (West Berlin, NJ). Where the wet strength is typically effected by wet strength additives such as kymene, cationic glyoxylated resins, polyamidoamine-epichlorohydrin resins, polyamine-epichlorohydrin resins, including epoxide resins. In embodiments, the formulation disclosed herein effects such wet strength in the absence of such additives.

As used herein “wet” means covered or saturated with water or another liquid.

in some embodiments, the processes disclosed herein may include a step of binding a specific SFAE to a cellulosic surface by contacting the cellulosic surface with formulation containing the specific SFAE. The processes may also include an additional step comprising exposing the contacted cellulose-based material to heat, radiation, a catalyst or a combination thereof for a sufficient time to bind the specific SFAE to the cellulose based material. In a related aspect, such radiation may include, but is not limited to UV, IR, visible light, or a combination thereof. In another related aspect, the reaction may be carried out at room temperature (i.e., 25° C.) to about 150° C., about 50° C. to about 100° C., or about 60° C. to about 80° C.

In embodiments, cellulosic material may be made lipophobic by the addition of polyvinyl alcohol (PvOH) and/or prolamines to the formulation. In one aspect, the prolamines include, for example, zein, gliadin, hordein, secalin, katirin and avenin. In a related aspect, the prolamine is zein.

In some embodiments, no catalysts and no organic carriers (e.g., volatile organic compounds) are required to carry out the binding reaction, including that no build-up of material is contemplated using the method as disclosed. In a related aspect, the reaction time is substantially instantaneous (i.e., less than 1 second). Further, the resulting material exhibits low blocking.

Using the formulations avoids the conventional use of, for example, chlorofluorocarbon, silicone, and petroleum based compounds to provide one or more of improved oil and grease resistance, water resistance, and gas and vapor barrier properties.

### The Specific SFAE

The formulations of the present disclosure employ a water insoluble and high melting point saccharide fatty acid ester (“the specific SFAE”) for imparting water resistance and/or oil and grease resistance (OGR) to cellulosic materials.

As used herein, the term “water insoluble” means that the specific SFAE does not dissolve in water at 25° C. For example, this may correspond to a solubility of 1000 mg/L or less.

The specific SFAE has a melting point higher than 100° C. In some aspects the melting point of the specific SFAE may be 110° C. or higher, 120° C. or higher, 130° C. or higher, 140° C. or higher, 150° C. or higher, or 150° C. or higher.

As explained above, higher melting point saccharide esters can be designed for higher temperature oil holdout applications.

The term “fatty acid” as used herein has its common meaning and refers to a carboxylic acid with an aliphatic chain, which may be saturated or unsaturated. The term fatty acid as used herein may refer to the fatty acid group bound to the saccharide by an ester bond (that is, one or more hydroxyl groups of the saccharide are esterified).

The fatty acid groups of the specific SFAE may be, for example, any known fatty acid. In preferred embodiments, the fatty acid is known to be present in food, is edible, and/or is approved by the FDA. In some embodiments, the fatty acid groups are obtained from oilseeds. In other embodiments, the fatty acids are obtained from other sources of naturally edible fats and oil.

The fatty acid groups can be independently selected from one or more saturated fatty acids, one or more monounsaturated fatty acids, and/or one or more polyunsaturated fatty acids. By independently, this means, for example, that a saccharide triester may include three different fatty acid groups.

Example saturated fatty acids for use in forming the specific SFAE (that is, for use when esterifying the saccharide moiety) include, for example, butyric acid (butanoic acid), caproic acid (hexanoic acid), caprylic acid (octanoic acid), capric acid (decanoic acid), lauric acid, (dodecanoic acid), myristic acid (tetradecanoic acid), palmitic acid (hexadecanoic acid), stearic acid (octadecanoic acid), arachidic acid (icosanoic acid), behenic acid (docosanoic acid), or lignoceric acid (tetracosanoic acid).

Example monounsaturated fatty acids for use in forming the specific SFAE include, for example, caproleic acid, (dec-9-enoic acid), lauroleic acid ((Z)-dodec-9-enoic acid), myristoleic acid ((Z)-tetradec-9-enoic acid), palmitoleic acid ((Z)-hexadec-9-enoic acid), oleic acid ((Z)-octadec-9-enoic acid), elaidic acid ((E)-octadec-9-enoic acid), vaccenic acid ((E)-octadec-11-enoic acid), gadoleic acid ((Z)-icos-9-enoic acid), erucic acid ((Z)-docos-13-enoic acid), brassidic acid ((E)-docos-13-enoic acid), or nervonic acid ((Z)-tetracos-15-enoic acid).

Example polyunsaturated fatty acids for use in forming the specific SFAE include, for example, linoleic acid (LA) ((9Z,12Z)-octadeca-9,12-dienoic acid), alpha-Linolenic acid (ALA) ((9Z,12Z,15Z)-octadeca-9,12,15-trienoic acid), gamma-Linolenic acid (GLA) ((6Z,9Z,12Z)-octadeca-6,9,12-trienoic acid), columbinic acid ((5E,9E,12E)-octadeca-5,9,12-trienoic acid), stearidonic acid ((6Z,9Z,12Z,1 5Z)-octadeca-6,9,12, 15-tetraenoic acid), mead acid ((5Z,8Z,11Z)-icosa-5,8,11-trienoic acid), dihomo-γ-linolenic acid (DGLA) ((8Z,11Z,14Z)-icosa-8,11,14-trieinoic acid), arachidonic acid ((5Z,8Z,11Z,14Z)-icosa-5,8,11,14-tetraenoic acid), eicosapentaenoic acid (EPA) ((5Z,8Z,11Z,14Z,17Z)-icosa-5,8,11,14,17-pentaenoic acid), docosapentaenoic acid (DPA) ((7Z,10Z,13Z,16Z,19Z)-docosa-7,10,13,16,19-pentaenoic acid), or docosahexaenoic acid (DHA) ((4G,7Z,10Z,13Z,16Z,19Z)-dacosa-4,7,10,13,16,19-hexaenoic acid).

In some embodiments, the melting point of the specific SFAE may be varied by modifying the fatty acid chains. For example, the addition of unsaturated side chains may reduce the melting point. In other words, the melting point of the specific SFAE can be modified by selection of the saccharide moiety and the selection of the fatty acid group(s).

In some embodiments, the specific SFAE has one or esterified hydroxyl groups of the saccharide moiety. In some aspects, the specific SFAE may be, for example, a monoester, diester, triester, or higher degree of substitution (e.g., pentaester). The specific SFAE may be, for example, a blend of specific SFAE having different degrees of substitution (e.g., a blend of specific SFAE containing, for example, saccharide monoesters, diesters, and triesters. In some aspects, the specific SFAE may have a monoester as the main component, a diester as the main component, or a triester as the main component.

Suitable saccharides for the SFAE may include, for example, a disaccharide, such as xylose, glucose, raffinose, maltodextrose, galactose, combinations of glucose, combinations of fructose, maltose, lactose, combinations of mannose, combinations of erythrose, isomaltose, isomaltulose, trehalose, trehalulose, cellobiose, laminaribiose, chitobiose, and combinations thereof.

As noted above, another suitable saccharides moiety is chitosan.

A person of skill in the art will appreciate that the water solubility of the SFAE can be selected by varying one or more of the parameters of the specific SFAE noted above. In this regard, when a plurality of the specific SFAE are used, each specific SFAE may have be chosen to have similar or different properties, such as, for example, similar or different HLB values (e.g., a lower range used in combination with higher range).

In some embodiments, the formulation may contain only one or more of the specific SFAE (i.e., consists of the specific SFAE). In some embodiments, the formulation may consist essentially of the SFAE, wherein the basic and material property of the formulation is for importing the barrier properties described herein.

In some embodiments, the formulation may be obtained by dispersing the specific SFAE in water. In some embodiments, such a dispersion may consist essentially of the specific SFAE and the water.

In some embodiments, the formulation may be obtained by dissolving or dispersing the specific SFAE in an organic solvent.

In some embodiments, the formulation may be a liquid system consisting of the specific SFAE in a solution of the soluble SFAE (that is, the soluble SFAE acts as the solvent to provide the liquid system). As noted above, the soluble SFAE dissolves in water at 25° C.

When the formulation contains both the specific SFAE and the soluble SFAE, a weight ratio of the specific SFAE to the soluble SFAE can be from about 0.1 :99.9 to about 99:0.1, from about 10:90 to about 90:10, from about 20:80 to about 80:20, from about 35:65 to about 65:35, from about 40:60 to about 60:40, from about 45:55 to about 55:45, or about 50:50.

While not being bound by theory, the interaction between the specific SFAE and the cellulose-based material may be by ionic, hydrophobic, van der Waals interaction, or covalent bonding, or a combination thereof. In a related aspect, the specific SFAE binding to the cellulose-based material is substantially irreversible (e.g., using a glyceride or fatty acid salt comprising a combination of saturated and unsaturated fatty acids).

in some embodiments, the hydrophobic barrier property is imparted to the substrate by the specific SFAE in the absence of any secondary hydrophobes.

Further, at a sufficient concentration, the binding of the specific SFAE alone is enough to make the contacted substrate hydrophobic: i.e., hydrophobicity is achieved in the absence of the addition of waxes, rosins, resins, diketenes, shellacs, vinyl acetates, PLA, PEI, oils, other water repellant chemicals or combinations thereof (i.e., secondary hydrophobes), including that other properties such as, inter alia, strengthening, stiffing, and bulking of the cellulose-based material is achieved by specific SFAE binding alone.

One advantage of the present disclosure may be that multiple fatty acid chains are reactive with the cellulose. Without being bound by any theory, this is thought to give rise to a crosslinking network, leading to strength improvements in fibrous webs such as paper, paperboard, air-laid and wet-laid non-wovens, and textiles. This is typically not found in other sizing or hydrophobic treatment chemistries. The specific SFAE as disclosed herein may also generate/increase wet strength, a property absent when using many other water resistant chemistries.

In embodiments, the amount of the specific SFAE used to impart hydrophobicity depends on the form of the substrate (e.g., the form of the cellulose-based material) and the method of contacting the surface of the substrate.

In one aspect, when the specific SFAE is coated by a known method on a cellulose-based material, the specific SFAE may present, for example, at a coating weight of at least about 0.05 g/m² to about 1.0 g/m², about 1.0 g/m² to about 2.0 g/m², about 2 g/m² to about 3 g/m², or higher concentration on a surface of the cellulose-based material. In some embodiments, the specific SFAE may coat the entire outer surface of a cellulose-based material (e.g., coat an entire piece of paper or cellulose-containing article).

In another aspect, when the cellulose-based material is a solution containing cellulose fiber and the formulation is added on the wet end of the paper-making process, the specific SFAE may be present, for example, at a concentration of at least about 0.025% (wt/wt) of the total fiber present. In a related aspect, it may be present at about 0.05% (wt/wt) to about 0.1% (wt/wt), about 0.1% (wt/wt) to about 0.5% (wt/wt), about 0.5% (wt/wt) to about 1.0% (wt/wt), about 1.0% (wt/wt) to about 2.0% (wt/wt), about 2.0% (wt/wt) to about 3.0% (wt/wt), about 3.0% (wt/wt) to about 4.0% (wt/wt), about 4.0% (wt/wt) to about 5.0% (wt/wt), about 5.00%(wt/wt) to about 10% (wt/wt), about 10% (wt/wt) to about 50% (wt/wt) of the total fiber present. In a further related aspect, the amount of the specific SFAE may be equal to the amount of fiber present.

In other embodiments, a formulation may contain, for example, about 0.9% to about 1.0%, about 1.0% to about 5.0%, about 5.0 to about 10%, about 10% to about 20%, about 20% to about 30%, about 40% to about 50% of the specific SFAE by weight of the formulation (wt/wt).

In embodiments, a method of producing bulky, fibrous structures that retain strength even when exposed to water is disclosed. Generally fibrous slurries that are dried form dense structures that are easily broken down upon exposure to water. Formed fiber products made using the method as disclosed may include paper plates, drink holders (e.g., cups), lids, food trays and packaging that would be light weight, strong, and be resistant to exposure to water and other liquids.

In embodiments, the specific SFAE can be mixed with polyvinyl alcohol (PvOH) to produce sizing agents for water resistant coatings. A synergistic relationship between SFAEs and PvOH has previously been demonstrated. While it is known that PvOH is itself a good film former, and forms strong hydrogen bonds with cellulose, it is not very resistant to water, particularly hot water. PvOH may provide a rich source of OH groups for the specific SFAE to crosslink along the cellulose fibers, which increases the strength of paper, for example, particularly wet strength, and water resistance beyond what is possible with PvOH alone. A known crosslinking agent, such as, for example, a dialdehyde (e.g., glyoxal, glutaraldehyde, and the like) may also be used.

In other aspects, the effect of the specific SFAE can be enhanced by the addition of one or more soluble SFAE, such as described in the 959 patent, and/or one or more glycerides and/or fatty acid salts, such as described in the 999 publication.

An advantage of additionally using SFAEs is that they may limit hydrogen bonding between cellulosic fibers, increasing the space between them, thus, increasing bulk without substantially increasing weight.

When used in the formulations, the soluble SFAE may comprise or consist essentially of sucrose esters of fatty acids. Many methods are known and available for making or otherwise providing the SFAEs of the present invention, and all such methods are believed to be available for use within the broad scope of the present disclosure. For example, in certain embodiments it may be preferred that the fatty acid esters are synthesized by esterifying a saccharide with one or more fatty acid moieties obtained from oil seeds including but not limited to, soybean oil, sunflower oil, olive oil, canola oil, peanut oil, and mixtures thereof.

In embodiments, the soluble SFAE may comprise a saccharide moiety, including but not limited to a sucrose moiety, which has been substituted by an ester moiety at one or more of its hydroxyl hydrogens. In a related aspect, disaccharide esters for use in this disclosure can have the structure of Formula I of the 959 patent, which is incorporated herein by reference.

Suitable disaccharides for the soluble SFAE may include xylose, glucose, raffinose, maltodextrose, galactose, combinations of glucose, combinations of fructose, maltose, lactose, combinations of mannose, combinations of erythrose, isomaltose, isomaltulose, trehalose, trehalulose, cellobiose, laminaribiose, chitobiose, and combinations thereof.

The SFAEs can be produced in the manner disclosed in the 959 patent. For example, SFAEs may be made by esterification with substantially pure fatty acids by known processes of esterification. They can be prepared also by trans-esterification using saccharide and fatty acid esters in the form of fatty acid glycerides derived, for example, from natural sources, for example, those found in oil extracted from oil seeds, for example soybean oil. Trans-esterification reactions providing sucrose fatty acid esters using fatty acid glycerides are described, for example, in U.S. Pat. Nos. 3,963,699; 4,517,360; 4,518,772; 4,611,055; 5,767,257; 6,504,003; 6,121,440; and 6,995,232, and International Publication WO1992004361, herein incorporated by reference in their entireties

In embodiments, the cellulose-based material includes, but is not limited to, paper, paperboard, paper sheets, paper pulp, cups, boxes, trays, lids, release papers/liners, compost bags, shopping bags, shipping bags, bacon board, tea bags, insulating material, containers for coffee or tea, pipes and water conduits, food grade disposable cutlery, plates and bottles, screens for TV and mobile devices, clothing (e.g., cotton or cotton blends), bandages, pressure sensitive labels, pressure sensitive tape, feminine products, and medical devices to be used on the body or inside it such as contraceptives, drug delivery devices, container for pharmaceutical materials (e.g., pills, tablets, suppositories, gels, etc.), and the like. Also, the coating technology as disclosed may be used on furniture and upholstery, outdoors camping equipment and the like.

In one aspect, the coatings as described herein are resistant to pH in the range of between about 3 to about 9. In a related aspect, the pH may be from about 3 to about 4, about 4 to about 5, about 5 to about 7, about 7 to about 9.

In embodiments, a method for treating a surface of a cellulose containing (or cellulosic) material is disclosed including applying to the surface a composition containing an alkanoic acid derivative having the formula (II) or (III):

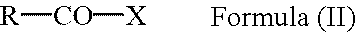

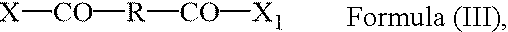

where R is a straight-chain, branched-chain, or cyclic aliphatic hydrocarbon radical having from 6 to 50 carbon atoms, and where X and X₁ are independently Cl, Br, R-COO-R, or O(CO)OR, where when the alkanoic acid derivative comprises formula (III) X or X₁ is the same or is different, where the specific SFAE as disclosed herein is a carrier, and where the method does not require an organic base, gaseous HCl, VOCs, or catalyst.

In one aspect, the formulation may also contain, for example, proteins, polysaccharides and/or lipids, including but not limited to, milk proteins (e.g., casein, whey protein and the like), wheat glutens, gelatins, prolamines (e.g., corn zein), soy protein isolates, starches, acetylated polysaccharides, alginates, carrageenans, chitosans, inulins, long chain fatty acids, waxes, and combinations thereof.

In embodiments, the specific SFAE and formulations of the present disclosure may be used, for example, to carry coatings or other chemicals used for paper manufacturing including but not limited to agalite, esters, diesters, ethers, ketones, amides, nitriles, aromatics (e.g., xylenes, toluenes), acid halides, anhydrides, talc, alkyl ketene dimer (AKD), alabaster, alganic acid, alum, albarine, glues, barium carbonate, barium sulfate, chlorine dioxide, clays, dolomite, diethylene triamine penta acetate, EDTA, enzymes, formamidine sulfuric acid, guar gum, gypsum, lime, magnesium bisulfate, milk of lime, milk of magnesia, polyvinyl alcohol (PvOH), rosins, rosin soaps, satins, soaps/fatty acids, sodium bisulfate, soda-ash, titania, surfactants, starches, modified starches, hydrocarbon resins, polymers, waxes, polysaccharides, proteins, and combinations thereof.

In some embodiments, the formulation may include one or more charged polymers to aid in the retention of the specific SFAE on the substrate. A charged polymer may aid in imparting the effects (e.g., barrier properties OGR and water resistance) by aligning the fatty acid groups of the specific SFAE.

The one or more charged polymers may include one or more cationic polymers, anionic polymers, nonionic polymers, and/or zwitterionic polymers. In some embodiments, a concentration of the cationic polymer in the formulation is from about 0.01% to about 5% by weight, from about 0.01% to about 3% by weight, 0.05% to about 0.1% by weight, or from about 0.1% to about 1% by weight, or from about 1% to about 3% by weight when a total weight of the formulation is considered 100%. In some aspects, a weight ratio in the formulation of the cationic polymer to the specific SFAE is from about 0.1:99.9 to about 20:80, from 0.5:99.5 to about 15:85, from about 1:99 to about 10:90, or from about 2.5:97.5 to about 7.5:92.5.

In some embodiments, the charged polymer has a weight average molecular weight of 500,000 to 10,000,000. In some embodiments, the weight average MW is 500,000 to 1,000,000, 1,000,001 to 2,000,000, 2,000,001 to 3,000,000 etc. In some embodiments, the charged polymer is a combination of two polymers having different weight average MW to achieve a bimodal-type blend.

Example cationic polymers for use as the retention aid may include, for example, polyacrylamide (e.g., polyDADMAC (poly diallyldimethylammonium chloride)), poly(ethyleneimine) (PEI), poly-1-(lysine) (PLL), poly[2-(N,N-dimethylamino)ethyl methacrylate] (PDMAEMA), and chitosan.

In embodiments, the treated cellulose-containing material (e.g., coated cellulose-containing material, or cellulose-containing material prepared by adding the formulation on the wet end) exhibits greater hydrophobicity or water-resistance relative to the cellulose-containing material without the treatment. In a related aspect, the treated cellulose-containing material exhibits greater lipophobicity or OGR relative to the cellulose-containing material without the treatment. In a further related aspect, the treated cellulose-containing material may be biodegradable, compostable, and/or recyclable. In one aspect, the treated cellulose-containing material is both hydrophobic (water resistant) and lipophobic (grease resistant).

In embodiments, the treated cellulose-containing material may have improved mechanical properties compared to that same material untreated. For example, paper bags treated by the process as disclosed herein show increased burst strength, Gurley Number, Tensile Strength and/or Energy of Maximum Load. In one aspect, the burst strength is increased by a factor of between about 0.5 to 1.0 fold, between about 1.0 and 1.1 fold, between about 1.1 and 1.3 fold, between about 1.3 to 1.5 fold. In another aspect, the Gurley Number increased by a factor of between about 3 to 4 fold, between about 4 to 5 fold, between about 5 to 6 fold and about 6 to 7 fold. In still another aspect, the Tensile Strain increased by a factor of between about 0.5 to 1.0 fold, between about 1.0 to 1.1 fold, between about 1.1 to 1.2 fold and between about 1.2 to 1.3 fold. And in another aspect, the Energy of Max Load increased by a factor of between about 1.0 to 1.1 fold, between about 1.1 to 1.2 fold, between about 1.2 to 1.3 fold, and between about 1.3 to 1.4 fold

In embodiments, the cellulose-containing material may be, for example, a base paper comprising microfibrillated cellulose (MFC) or cellulose nanofiber (CNF) as described for example in U.S. Pub. No. 2015/0167243 (herein incorporated by reference in its entirety), where the MFC or CNF is added during the forming process and paper making process and/or added as a coating or a secondary layer to a prior forming layer to decrease the porosity of said base paper. In a related aspect, the base paper is contacted with the formulations as described above.

In a further related aspect, the contacted base paper may, for example, be further contacted with a polyvinyl alcohol (PvOH). In embodiments, the resulting contacted base paper is tuneably water and lipid resistant. In a related aspect, the resulting base paper may exhibit a Gurley value of at least about 10-15 (i.e., Gurley Air Resistance (sec/100 cc, 20 oz. cyl.)), or at least about 100, at least about 200 to about 350. In one aspect, the formulation may be a laminate for one or more layers or may provide one or more layers as a laminate or may reduce the amount of coating of one or more layers to achieve the same performance effect (e.g., water resistance, OGR, and the like). In a related aspect, the laminate may comprise a biodegradable and/or composable heat seal or adhesive.

In embodiments, the specific SFAE may be combined with one or more coating components for internal and surface sizing (alone or in combination), including but not limited to, pigments (e.g., clay, calcium carbonate, titanium dioxide, plastic pigment), binders (e.g., starch, soy protein, polymer emulsions, PvOH, casein), and additives (e.g., glyoxal, glyoxalated resins, zirconium salts, polyethylene emulsion, carboxymethyl cellulose, acrylic polymers, alginates, polyacrylate gums, polyacrylates, microbiocides, oil based defoamers, silicone based defoamers, stilbenes, direct dyes and acid dyes). In a related aspect, such components may provide one or more properties, including but not limited to, building a fine porous structure, providing light scattering surface, improving ink receptivity, improving gloss, binding pigment particles, binding coatings to paper, base sheet reinforcement, filling pores in pigment structure, reducing water sensitivity, resisting wet pick in offset printing, preventing blade scratching, improving gloss in supercalendering, reducing dusting, adjusting coating viscosity, providing water holding, dispersing pigments, maintaining coating dispersion, preventing spoilage of coating/coating color, controlling foaming, reducing entrained air and coating craters, increasing whiteness and brightness, and controlling color and shade. It will be apparent to one of skill in the art that combinations may be varied depending on the property(ies) desired for the final product.

In embodiments, the methods employing the formulations comprising the specific SFAE may be used to lower the cost of applications of primary/secondary coating (e.g., silicone-based layer, starch-based layer, clay-based layer, PLA-layer, PEI-layer and the like) by providing a layer of material that exhibits a necessary property (e.g., water resistance, low surface energy, and the like), thereby reducing the amount of primary/secondary layer necessary to achieve that same property. In one aspect, materials may be coated on top of a specific SFAE layer (e.g., heat sealable agents). In embodiments, the composition is fluorocarbon and silicone free.

In embodiments, the formulations increase both mechanical and thermal stability of the treated product. In one aspect, the surface treatment is thermostable at temperatures between about -100° C. to about 300° C. In further related aspect, the surface of the treated substrate (e.g., a cellulose-based material) exhibits a water contact angle of between about 60° to about 120°. In another related aspect, the surface treatment is chemically stable at temperatures of between about 200° C. to about 300° C.

The substrate, which may be dried prior to application (e.g., at about 80-150° C.), may be treated with the modifying formulations comprising the specific SFAE by dipping, for example, and allowing the surface to be exposed to the composition for less than 1 second. The substrate may be heated to dry the surface, after which the modified material is ready for use. In one aspect, according to the method as disclosed herein, the substrate may be treated by any suitable coating/sizing process typically carried out in a paper mill (see, e.g., Smook, G., Surface Treatments in Handbook for Pulp & Paper Technologists, (2016), 4^{th} Ed., Cpt. 18, pp. 293-309, TAPPI Press, Peachtree Corners, GA USA, herein incorporated by reference in its entirety).

No special preparation of the cellulose-containing material is necessary in practicing this disclosure, although for some applications, the material may be dried before treatment. In embodiments, the methods as disclosed may be used on any cellulose-based surface, including but not limited to, a film, a rigid container, fibers, pulp, a fabric or the like. In one aspect, the formulation may be applied by conventional size press (vertical, inclined, horizontal), gate roll size press, metering size press, calender size application, tube sizing, on-machine, off-machine, single-sided coater, double-sided coater, short dwell, simultaneous two-side coater, blade or rod coater, gravure coater, gravure printing, flexographic printing, ink-jet printing, laser printing, supercalendering, and combinations thereof.

Depending on the source, the cellulose treated in the methods herein may be paper, paperboard, pulp, softwood fiber, hardwood fiber, or combinations thereof, nanocellulose, cellulose nanofibres, whiskers or microfibril, microfibrillated, cotton or cotton blends, cellulose nanocrystals, or nanofibrilated cellulose.

In addition, fibers and cellulose-based material modified as disclosed herein, may be repulped. Further, for example, water cannot be easily “pushed” past the low surface energy barrier into the sheet.

In embodiments, the amount of the formulation is sufficient to completely cover at least one surface of a substrate, such as at least one surface of a cellulose-containing material. For example, in embodiments, the formulation may be applied to the entire outer surface of a container, such as a container for foodstuffs, the complete inner surface of a container, or a combination thereof, or one or both sides of a base paper. In other embodiments, the complete upper surface of a film may be covered by the formulation, or the complete under surface of a film may be covered by the formulation, or a combination thereof. In some embodiments, the lumen of a device/instrument may be covered by the coating or the outer surface of the device/instrument may be covered by formulation, or a combination thereof.

In embodiments, the amount of formulation applied is sufficient to partially cover at least one surface of a cellulose-containing material. For example, only those surfaces exposed to the ambient atmosphere may be covered by the formulation, or only those surfaces that are not exposed to the ambient atmosphere are covered by the formulation (e.g., masking). As will be apparent to one of skill in the art, the amount of formulation applied may be dependent on the use of the material to be covered. In one aspect, one surface may be coated with the formulation and the opposing surface may be coated with an agent including, but not limited to, proteins, wheat glutens, gelatins, prolamines, soy protein isolates, starches, modified starches, acetylated polysaccharides, alginates, carrageenans, chitosans, inulins, long chain fatty acids, waxes, and combinations thereof. In a related aspect, the formulation can be added to a furnish, and the resulting material on the web may be provided with an additional coating of glycerides/fatty acid salts.

Any suitable coating process may be used to deliver any of the various formulations in the course of practicing this aspect of the method. In embodiments, a processes of coating the formulations may include, for example, immersion, spraying, painting, printing, and any combination of any of these processes, alone or with other coating processes adapted for practicing the methods as disclosed.

By increasing the concentration of the specific SFAE, for example, the composition as disclosed herein may react more extensively with the substrate (e.g., cellulose) being treated with the net result that again improved water-repellent/lipid resistance characteristics are exhibited. However, higher coat weights do not necessarily translate to increased water resistance. In one aspect, various catalysts might allow for speedier “curing” to precisely tune the quantity of the specific SFAE to meet specific applications

It will be apparent to one of skill in the art that, outside of any particular ranges or compositions described in detail herein, the selection of a cellulose to be treated, the specific SFAE, the reaction temperature, and the exposure time are process parameters that may be optimized by routine experimentation to suit any particular application for the final product.

The derivatized cellulose-based materials described herein have altered physical properties which may be defined and measured using appropriate tests known in the art. For hydrophobicity the analytical protocol may include, but is not limited to, the contact angle measurement and moisture pick-up. Other properties include, stiffness, WVTR, porosity, tensile strength, lack of substrate degradation, burst and tear properties. A specific standardized protocol to follow is defined by the American Society for Testing and Materials (protocol ASTM D7334 - 08).

The permeability of a surface to various gases such as water vapour and oxygen may also be altered by the specific SFAE process as the barrier function of the material is enhanced. The standard unit measuring permeability is the Barrer and protocols to measure these parameters are also available in the public domain (ASTM std F2476-05 for water vapour and ASTM std F2622-8 for oxygen).

In embodiments, materials treated according to the disclosed methods display a complete biodegradability as measured by the degradation in the environment under microorganismal attack.

Various methods are available to define and test biodegradability including the shake-flask method (ASTM E1279 - 89(2008)) and the Zahn-Wellens test (OECD TG 302 B).

Various methods are available to define and test compostability including, but not limited to, ASTM D6400.

Cellulosic materials suitable for treatment by the processes of this disclosure include but are not limited to cotton fibers, plant fibers such as flax, wood fibers, regenerated cellulose (rayon and cellophane), partially alkylated cellulose (cellulose ethers), partially esterified cellulose (acetate rayon), and other modified cellulose materials which have a substantial portion of their surfaces available for reaction/binding. As stated above, the term “cellulose” includes all of these materials and others of similar polysaccharide structure and having similar properties. Among these the relatively novel material microfibrillated cellulose (cellulose nanofiber) (see e.g., U.S. Pat. No. 4,374,702 and U.S. Pat. Application Publication Nos. 2015/0167243 and 2009/0221812, herein incorporated by reference in their entireties) is particularly suitable for this application. In other embodiments, celluloses may include but are not limited to, cellulose triacetate, cellulose propionate, cellulose acetate propionate, cellulose acetate butyrate, nitrocellulose (cellulose nitrate), cellulose sulfate, celluloid, methylcellulose, ethylcellulose, ethyl methyl cellulose, hydroxyethyl cellulose, hydroxypropyl cellulose, cellulose nanocrystals, hydroxyethyl methyl cellulose, hydroxypropyl methyl cellulose, ethyl hydroxyethyl cellulose, carboxymethyl cellulose, and combinations thereof.

The modification of the cellulose as disclosed herein, in addition to increasing its hydrophobicity, may also increase its tensile strength, flexibility and stiffness, thereby further widening its spectrum of use. All biodegradable and partially biodegradable products made from or by using the modified cellulose disclosed in this application are within the scope of the disclosure, including recyclable and compostable products.

Among the possible applications of the coating technology disclosed herein such items include, but are not limited to, containers for all purpose such as paper, paperboard, paper pulp, cups, lids, boxes, trays, release papers/liners, compost bags, shopping bags, pipes and water conduits, food grade disposable cutlery, plates and bottles, screens for TV and mobile devices, clothing (e.g., cotton or cotton blends), bandages, pressure sensitive labels, pressure sensitive tape, feminine products, and medical devices to be used on the body or inside it such as contraceptives, drug delivery devices, and the like. Also, the coating technology as disclosed may be used on furniture and upholstery, outdoors camping equipment and the like.

### EXAMPLES

In the following, although embodiments of the present disclosure are described in further detail by means of Examples, the present disclosure is not limited thereto.

### Example 1

For Example 1, specific SFAEs were prepared and melting points were measured, as shown in Table 1 below.

TABLE 1

| specific SFAE | Melting Point C | Solubility in Water at 25° C. |
|---|---|---|
| lactose monostearate | ∼114 onset | Insoluble |
| maltose monostearate | ∼135 onset | Insoluble |
| raffinose monostearate | ∼152 Decomposition | soluble |
| lactose monolaurate | > 106 | Insoluble |
| maltose monolaurate | > 159 | Insoluble |
| raffinose monolaurate | 169-174 | soluble |
| trehalose distearate | 145-160 | Insoluble |
| trehalose monostearate | 128-130 | Insoluble |
| trehalose dipalmitate | 145-162 | Insoluble |
| trehalose monopalmitate | 114-116 | Insoluble |
| trehalose dimyristate | 157-161 | Insoluble |
| trehalose monomyristate | 114-116 | Insoluble |
| trehalose dilaurate | 161-164 | Insoluble |
| trehalose monolaurate | 156-158 | soluble |

### Example 2

For Example 2. several specific SFAE were tested to determine their ability to impart a barrier property on a base paper.

A Southworth paper was chosen as a base paper. The base paper was approximately a 40# sheet with a Gurley porosity running around 200 sec.

Three different specific SFAE were prepared, dissolved in an organic solvent (a solution of 70% ethanol, and 30% chloroform), and applied to the base paper via hand drawdown at the coating weight listed in Table 2. The coating weight is the weight (pounds/ton) is the weight of the specific SFAE after the organic solvent was evaporated. Once the papers were dried and conditioned, the SFAE coated base papers were tested for water resistance and lipid resistance (OGR) using various tests, and compared to uncoated base paper as a control.

Water resistance was tested using a water Cobb test adapted from Tappi Standard Test Method T 441 om-20 “Water Absorptiveness of Paper.”

Oil and grease resistance was tested using several tests:
(i) a 3 M KIT Test (Tappi Standard Test Method T 559 “Grease resistance”),
(ii) a Folded KIT Test, wherein the paper is subjected to a 180° fold, reopoend, and then grease is placed on the resulting crease according to the same 3 M KIT Test.
(iii) oil Cobb tests using vegetable oil adapted from Tappi Standard Test Method T 441 om-20 at room temperature of about 20-23° C. and at 100° C.; and
(iv) an Oil Drop test in a 100° C. oven.

The data in Table 2 shows, for example, that when the pores of the base paper are filled with the tested specific SFAE varieties, the base paper was capable of holding oil out when it is hot.

The results demonstrate that the specific SFAE can address the problem of migration of barrier formulations into food or packaged products, particularly at high temperatures.

TABLE 2

| Southworth Base | Coat Weight/ton | Water Cobb (2 min) | KIT | Folded KIT | Oil Cobb (50 sec) | 100° C. Oil Cobb (60 sec) | Oil Drop in 100° C. Oven |
|---|---|---|---|---|---|---|---|
| Basepaper | | 24 | Fail <3 | Fall <3 | 23.2 | 20 | Fail Instantly <1 sec |
| Trehalose Distearate | 122 | 24.8 | Pass 6 Fail 7 | Pass 3 Fail 4 | 4.8 | 6.4 | Fail at 2:00 min |
| Trehalose Dilaurate | 125 | 44 | Pass 6 PH 7 Fail 8 | Pass 3 Fail 3 | 3.2 | 4.8 | Fail at 8:12 min |
| Trehalose Dipalmitate | 109 | 32.8 | Pass 5 PH 6 Fail 7 | Fail <3 | 4 | 14.4 | Fail at 6:30 min |

While there have been shown and described fundamental novel features of the disclosure as applied to the preferred and exemplary embodiments thereof, it will be understood that omissions and substitutions and changes in the form and details of the disclosure may be made by those skilled in the art without departing from the spirit of the disclosure. Moreover, as is readily apparent, numerous modifications and changes may readily occur to those skilled in the art. For example, any feature(s) in one or more embodiments may be applicable and combined with one or more other embodiments. Hence, it is not desired to limit the present disclosure to the exact construction and operation shown and described and, accordingly, all suitable modification equivalents may be resorted to falling within the scope of the present disclosure as claimed. In other words, although the embodiments of the disclosure have been described with reference to the above examples, it will be understood that modifications and variations are encompassed within the spirit and scope of the disclosure. Accordingly, the invention is limited only by the following claims.

All references disclosed herein are hereby incorporated by reference in their entireties.

What is claimed is:

1. A method of imparting a barrier property to a cellulose-based substrate, the method comprising:
preparing a formulation for imparting the barrier property, the formulation comprising a specific saccharide fatty acid ester (specific SFAE); and
contacting a surface of the cellulose-based substrate with the formulation to impart the barrier property to the cellulose-based substrate,
wherein the barrier property is increased water resistance and/or increased lipid resistance,
the specific SFAE is insoluble in water at 25° C., and
the specific SFAE has a melting point higher than 100° C.

2. The method of claim 1, wherein the step of contacting comprises forming a slurry of the formulation and cellulose fiber as the cellulose-based substrate.

3. The method of claim 2, wherein the specific SFAE is present in the slurry at a total concentration of at least 0.025% (wt/wt) of the total cellulose fiber present in the slurry.

4. The method claim 2, further comprising forming a solid article after draining the cellulose fiber from the slurry, the solid article possessing the barrier property.

5. The method of claim 4, wherein the article is selected from the group consisting of paper, paperboard, bacon board, insulating material, a carton for food storage, a compost bag, a bag for food storage, release paper, a shipping bag, weed-block/barrier fabric or film, mulching film, plant pots, packing beads, bubble wrap, laminates, envelopes, gift cards, credit cards, gloves, raincoats, OGR paper, a shopping bag, diapers, membranes, eating utensil, a tea bag, a container for coffee or tea, a container for holding hot or cold beverages, a cup, a plate, a bottle for carbonated liquid storage, a bottle for non-carbonated liquid storage, a lid, film for wrapping food, a garbage disposal container, a food handling implement, a fabric fibre, a water storage and conveying implement, a storage and conveying implement for alcoholic or non-alcoholic beverages, an outer casing or screen for electronic goods, an internal or external piece of furniture, a curtain, upholstery, fabric, film, a box, a sheet, a tray, a pipe, a tube, a water conduit, clothing, a medical device, pharmaceutical packaging, a contraceptive, camping equipment, cellulosic material that is molded, and combinations thereof.

6. The method of claim 1, wherein the step of contacting comprises coating the surface of the cellulose-based substrate with the formulation.

7. The method of claim 6, wherein the specific SFAE is present at a weight of at least 0.05 g/m² on the surface of the substrate.

8. The method claim 6, wherein the cellulose-based substrate is an article selected from the group consisting of paper, paperboard, bacon board, insulating material, paper pulp, a carton for food storage, a compost bag, a bag for food storage, release paper, a shipping bag, weed-block/barrier fabric or film, mulching film, plant pots, packing beads, bubble wrap, oil absorbent material, laminates, envelops, gift cards, credit cards, gloves, raincoats, OGR paper, a shopping bag, diapers, membranes, eating utensil, a tea bag, a container for coffee or tea, a container for holding hot or cold beverages, a cup, a plate, a bottle for carbonated liquid storage, a bottle for non-carbonated liquid storage, a lid, film for wrapping food, a garbage disposal container, a food handling implement, a fabric fibre, a water storage and conveying implement, a storage and conveying implement for alcoholic or non-alcoholic beverages, an outer casing or screen for electronic goods, an internal or external piece of furniture, a curtain, upholstery, fabric, film, a box, a sheet, a tray, a pipe, a tube, a water conduit, clothing, a medical device, pharmaceutical packaging, a contraceptive, camping equipment, cellulosic material that is molded, and combinations thereof.

9. The method of claim 1, wherein the barrier property imparted to the cellulose-based substrate is increased lipid resistance, and the increased lipid resistance is provided by the specific SFAE in the in the absence of any secondary hydrophobes.

10. The method of claim 1, wherein the formulation further comprises a soluble saccharide fatty acid ester (soluble SFAE) which is soluble in water at 25° C.

11. The method of claim 10, wherein the formulation is a liquid system consisting essentially of the specific SFAE and the soluble SFAE.

12. The method of claim 1, wherein the formulation further comprises one or more glycerides and/or one or more fatty acid salts.

13. The method of claim 1, wherein the cellulose-based substrate imparted with the barrier property exhibits a 3 M grease KIT test value of between about 3 and about 12.

14. The method of claim 1, wherein the surface of the cellulose-based substrate imparted with the barrier property exhibits a water contact angle greater than 90°.

15. The method of claim 1, wherein the surface of the cellulose-based substrate imparted with the barrier property exhibits an HST value of at least 65 secs.

16. The method of claim 1, wherein the specific SFAE has a melting point higher than 125° C.

17. The method of claim 1, wherein the saccharide moiety of the specific SFAE is selected from lactose, maltose, raffinose, or trehalose.

18. The method of claim 1, wherein the fatty acid group(s) of the specific SFAE are one of more selected from stearate, laurate, myristate, or palmitate.

19. The method of claim 1, wherein the specific SFAE includes a blend of one or more saccharide monoesters and saccharide diesters.

20. The method of claim 1, wherein the saccharide moiety of the specific SFAE is chitosan.

21. An article obtained by the method according to any preceding claim.

22. The article according to claim 21, wherein the article is selected from the group consisting of paper, paperboard, bacon board, insulating material, a carton for food storage, a compost bag, a bag for food storage, release paper, a shipping bag, weed-block/barrier fabric or film, mulching film, plant pots, packing beads, bubble wrap, laminates, envelops, gift cards, credit cards, gloves, raincoats, OGR paper, a shopping bag, diapers, membranes, eating utensil, a tea bag, a container for coffee or tea, a container for holding hot or cold beverages, a cup, a plate, a bottle for carbonated liquid storage, a bottle for non-carbonated liquid storage, a lid, film for wrapping food, a garbage disposal container, a food handling implement, a fabric fibre, a water storage and conveying implement, a storage and conveying implement for alcoholic or non-alcoholic beverages, an outer casing or screen for electronic goods, an internal or external piece of furniture, a curtain, upholstery, fabric, film, a box, a sheet, a tray, a pipe, a tube, a water conduit, clothing, a medical device, pharmaceutical packaging, a contraceptive, camping equipment, cellulosic material that is molded, and combinations thereof.

23. The article of claim 22, wherein a surface of the article exhibits a 3 M grease KIT test value of between about 3 and about 12.

24. The article of claim 22, wherein a surface of the article exhibits a water contact angle greater than 90°.

25. The article of claim 22, wherein a surface of the article exhibits an HST value of at least 65 secs.

26. A formulation for imparting a barrier property to a cellulose-based substrate, the formulation comprising a specific saccharide fatty acid ester (specific SFAE),
wherein the barrier property is increased water resistance and/or increased lipid resistance,
the specific SFAE is insoluble in water at 25° C., and
the specific SFAE has a melting point higher than 100° C., and
the specific SFAE is present in the formulation in an amount sufficient to impart the barrier property to the substrate.

27. The formulation of claim 26, wherein the formulation further comprises a soluble saccharide fatty acid ester (soluble SFAE) which is soluble in water at 25° C.

28. The formulation of claim 27, wherein the formulation is a liquid system consisting essentially of the specific SFAE and the soluble SFAE.

29. The formulation of claim 26, wherein the formulation further comprises one or more glycerides and/or one or more fatty acid salts.

30. The formulation of claim 26, wherein the specific SFAE has a melting point higher than 125° C.

31. The formulation of claim 26, wherein the saccharide moiety of the specific SFAE is selected from lactose, maltose, raffinose, or trehalose.

32. The formulation of claim 26, wherein the fatty acid group(s) of the specific SFAE are one of more selected from stearate, laurate, myristate, or palmitate.

33. The formulation of claim 26, wherein the specific SFAE includes a blend of one or more saccharide monoesters, saccharide diesters, and/or saccharide triesters.

34. The formulation of claim 26, wherein the saccharide moiety of the specific SFAE is chitosan.
